(19) United States
(12) Patent Application Publication
(10) Pub. No.: US 2016/0362242 A1
(43) Pub. Date: Dec. 15, 2016

(54) DISPENSING SYSTEM FOR DISPENSING OF DISPOSABLE GLOVES

(71) Applicant: Foodhandler, Inc., Reno, NV (US)

(72) Inventor: Jian Tao, Reno, NV (US)

(21) Appl. No.: 15/177,516

(22) Filed: Jun. 9, 2016

Related U.S. Application Data
(60) Provisional application No. 62/173,627, filed on Jun. 10, 2015.

(51) Int. Cl. B65D 83/08

(52) U.S. Cl. CPC B65D 83/0805; B65D 83/0894; A61B 42/40

(57) ABSTRACT

A dispensing system for the dispensing of single use disposable gloves is provided. The device may be disposable or non-disposable. The device includes an opening through which the disposable gloves are dispensed in a non-interleaved fashion. When gloves are dispensed from the dispensing system disclosed herein, the gloves can be dispensed cuff-first in order to reduce the risk of contamination and/or desterilization.

### CROSS-REFERENCE TO RELATED APPLICATIONS

This application claims the benefit of U.S. Provisional Application No. 62/173,627, filed Jun. 10, 2015, which is hereby incorporated by reference in its entirety.

### BACKGROUND OF THE INVENTION

Single use disposable gloves are used for a variety of purposes and in a number of different industries. For example, single use disposable gloves may be used in warehouse facilities to protect workers from injury or harmful chemicals, in hospitals and other healthcare facilities to preserve a sterile field, and in the food preparation industry to prevent contamination in foods. Typically, single use disposable gloves are made of a variety of materials such as polyethylene, polyvinyl chloride, natural rubber latex, nitrile, and other materials.

For all of the above disposable glove types, the most common packaging used for dispensing the gloves is a disposable paperboard box. Often that paperboard box may be placed within a carton outer case designed for transportation and storage of the gloves. Typically the paperboard dispensing box is discarded when empty and replaced by another stocked dispensing box within the carton outer case.

Existing paperboard dispensing boxes work well in most environments. However, under certain circumstances, for example when a working environment is wet, a regular paperboard dispensing box may become saturated and subsequently lose strength. As a result, unused gloves inside the box are likely to become unsterile and/or contaminated. Therefore, the boxes are often thrown away prior to all of the gloves being used as a result of the gloves becoming unsterile and/or contaminated. This increases environmental and monetary waste.

Existing paperboard boxes occupy substantial space due to their rigid rectangular shape. The rigid shape of such paperboard boxes is largely dictated by the dispensing function of the paperboard boxes. Traditionally, paperboard boxes for dispensing disposable gloves are perforated at a center top surface as illustrated in the traditional paperboard box shown in prior art FIG. 1.

FIG. 1 illustrates a dispensing device 10 having a center dispensing portion 11. Dispensing device 10 serves its intended use but has a number of drawbacks. When a glove is dispensed from dispensing device 10, a user's fingers (which could be contaminating) could touch any part of a glove being dispensed from dispensing device 10, not just the glove's cuff. A contaminating finger touching an otherwise sterile glove dramatically increases the likelihood that gloves could be unsterilized and/or contaminated prior to donning by a wearer.

Prior art solutions exist where gloves are packed in an interleaved fashion, to ensure that only the cuff of the glove is touched when a glove is dispensed. However, in those prior art solutions, a special apparatus is used in the packaging in order to dispense those interleaved gloves.

### SUMMARY OF THE PRESENT INVENTION

The present invention relates to a dispensing system for dispensing disposable gloves therefrom. The dispensing system includes a dispensing device that preferably includes each of a right, left, front, and rear side wall, as well as a top member. The dispensing device may have a substantially rectangular shape or other oval shape.

At least part of the top member of the dispensing device preferably includes an opening through which disposable gloves may be dispensed. The disposable gloves are contained within the dispensing device such that they are stacked one on top of another, and not in an interleaved fashion. When the gloves are dispensed through the opening as positioned according to the teachings hereof, a user can grasp the glove by the cuff only. This decreases the likelihood that the gloves are contaminated prior to being used.

In at least one embodiment, the dispensing system also includes a disposable packaging within the dispensing device. The disposable packaging contains the disposable gloves and can simply be replaced when empty. The disposable packaging is preferably made of a waterproof material, and may be a packaging such as a plastic bag. If the dispensing system is made from waterproof materials such as but not limiting to plastic, stainless steel, then the disposable packaging doesn't have to be waterproof such as nonwoven, paper, etc.

In another embodiment, the dispensing system further may include a door member hingedly attached to the top portion of the dispensing device. The door member may cover the opening when the dispensing device is not in use in order to reduce the likelihood of the disposable gloves becoming contaminated. The door member may be automatically controlled so that a user may open the door by waving his or her hand in front of the door. Alternatively, the door may be manually controlled or controlled by simple activation means like a switch or button.

In alternative embodiments, the opening may be located in a number of locations. It may be located on part of the top member of the dispensing device and either of the front side wall and the rear wall. In another embodiment, the opening may be located on part of the top member and at least part of one of the right side wall and the left side wall. In yet another embodiment, the opening is provided at a portion of the top member as well as part of one of the right or left side walls and the front or rear side walls.

The dispensing device may be disposable or non-disposable. In the non-disposable embodiment, the dispensing device is preferably made up of at least one of: plastic, metal, wood, rubber, or leather.

### BRIEF DESCRIPTION OF THE DRAWINGS

In the accompanying drawings, which form a part of the specification and are to be read in conjunction therewith in which like reference numerals are used to indicate like or similar parts in the various views:

FIG. 1 is a perspective view of a prior art dispensing device for dispensing disposable gloves;

FIG. 2 is a perspective view of a first embodiment of a dispensing device for dispensing disposable gloves according to the teachings of the present invention;

FIG. 3 is a first alternative embodiment of the dispensing device of FIG. 2;

FIG. 4 is a second alternative embodiment of the dispensing device of FIG. 2; and

FIG. 5 is a third alternative embodiment of the dispensing device of FIG. 2.

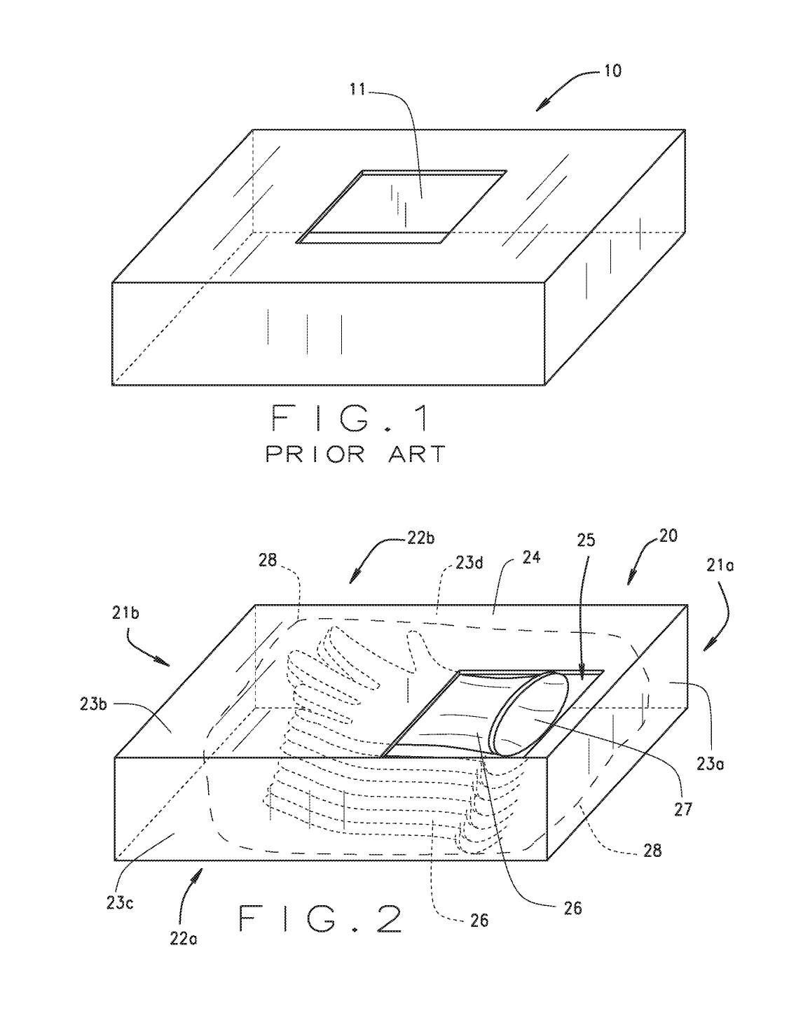

### DETAILED DESCRIPTION OF THE INVENTION

The present invention provides a dispensing device and associated components for dispensing disposable gloves in a non-interleaved fashion. FIG. 2 illustrates a first embodiment of a dispensing device 20 for dispensing disposable gloves therefrom. Dispensing device 20 includes each of a right portion 21a and left portion 21b, as well as a front portion 22a and a rear portion 22b. Dispensing device 20 also preferably includes side wall members 23 including right, left, front, rear side wall members 23a, 23b, 23c, 23d being located at each of portions 21a, 21b, 22a, 22b on dispensing device 20. Dispensing device 20 is generally rectangular in shape, and thus it includes four side wall members 23. A top member 24 of dispensing device 20 may also include an opening 25, for dispensing gloves 26 therefrom. As shown in FIG. 2, because opening 25 is located at right portion 21a of top member 24 of dispensing device 20, a user may remove gloves 26 by only touching cuffs 27 of gloves 26.

In dispensing device 20 (and the dispensing devices described herein below), opening 25 may be located similarly at a left portion 21b or any other portion of a dispensing device so long as gloves associated therewith can be dispensed “cuff-first.” Because a user may remove gloves 26 by only touching cuffs 27 when using dispensing device 20, gloves 26 may be removed from dispensing device 20 with a reduced risk of glove 26 contamination.

Dispensing device 20 may either be used as a single use disposable system or a system which may be refillable and thus be able to be used over and over again. In the embodiment where a dispensing device is disposable, a dispensing device may be made of a simple plastic film or other waterproof material, fabric with waterproof coating, structured plastic film with aluminum lamination, plastic and paper laminated films, etc.

The dispensing device as of FIG. 2 may be reusable. In the illustrated embodiment of FIG. 2, dispensing device 20 is preferably made of a waterproof material, for example plastic, metal, wood, rubber, or leather. Disposable packaging 28 is provided within dispensing device 20 in the illustrated embodiment in order to contain gloves 26. Disposable packaging 28 is preferably a plastic bag, though in alternative embodiments may be made of a simple plastic film or other waterproof material, fabric with waterproof coating, structured plastic film with aluminum lamination, plastic and paper laminated films, etc.

Gloves 26 within disposable packaging 28 may be packed more efficiently for storage and transportation than the traditional paperboard box packaging. Disposable packaging 28 may be used to contain many more gloves in the same volume as regular paperboard boxes. When gloves 26 of disposable packaging 28 are depleted, a user may simply replace disposable packaging 28 within dispensing device 20 with new disposable packaging 28 stocked with disposable gloves.

As shown in FIG. 2, gloves 26 are stacked in one on top of another within disposable packaging 28, all facing in the same direction. This is in contrast to the interleaved manner in which gloves were stacked in previous dispensing devices such as prior art dispensing device 10. Gloves 26 stacked on top of one another all facing the same direction as shown in FIG. 2 reduces the need for an additional apparatus to dispense gloves 26. Such an apparatus would be necessary in dispensing device 10 to dispense gloves in a “cuff-first” fashion when the hole is at the middle of the top.

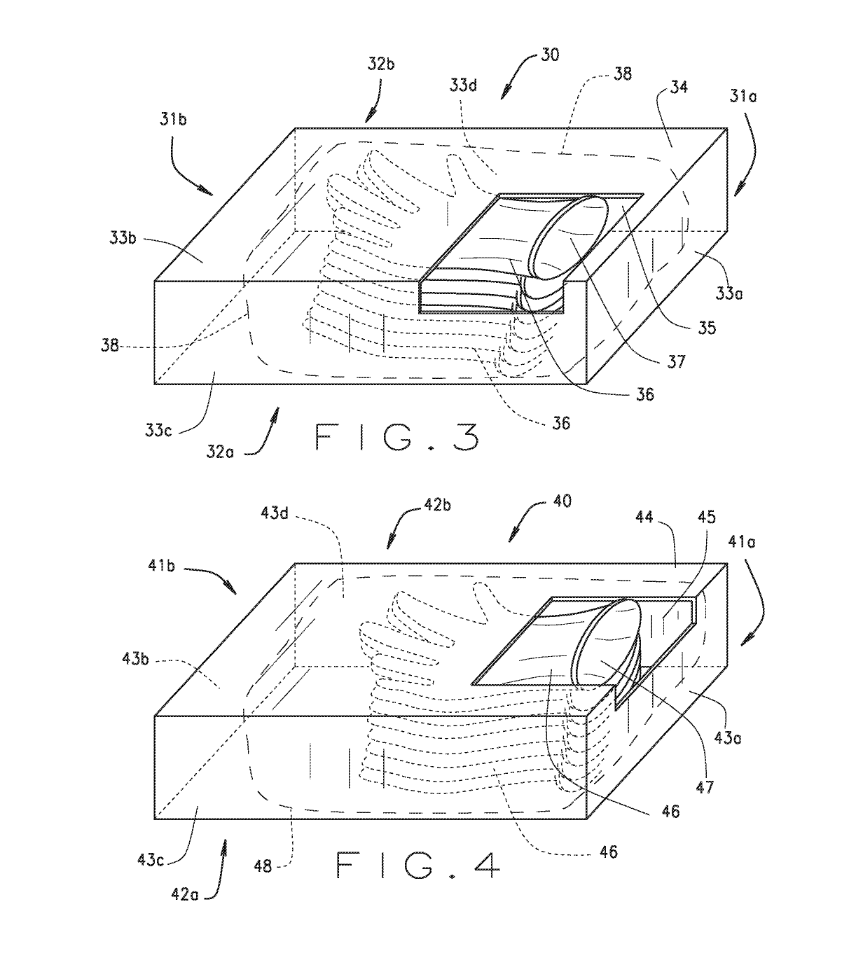

FIG. 3 illustrates a first alternative dispensing device 30 which is substantially similar in size, shape, and material to dispensing device 20. Like dispensing device 20, dispensing device 30 includes right and left end portions 31a and 31b, respectively, and front and rear portions 32a and 32b, respectively. Dispensing device 30 includes four side wall members 33: 33a, 33b, 33c, 33d located at portions 31a, 31b, 32a, and 32b, respectively. Additionally, dispensing device 30 may further include a top member 34. An opening 35 of dispensing device 30 is positioned such that it occupies right side portion 31a of top member 34 and the front side portion 32a of side wall 33 of dispensing device 30.

This particular positioning of opening 35 allows for easier access to gloves 36 of opening 35 when the supply of gloves 36 is getting low. Like dispensing device 20, gloves 36 of dispensing device 30 may be dispensed such that a user may only need to touch cuffs 37 of gloves 36.

Like dispensing device 20, dispensing device 30 may be either disposable or non-disposable. As shown in FIG. 3, disposable packaging 38 is provided for containing gloves 36 in a non-interleaved fashion.

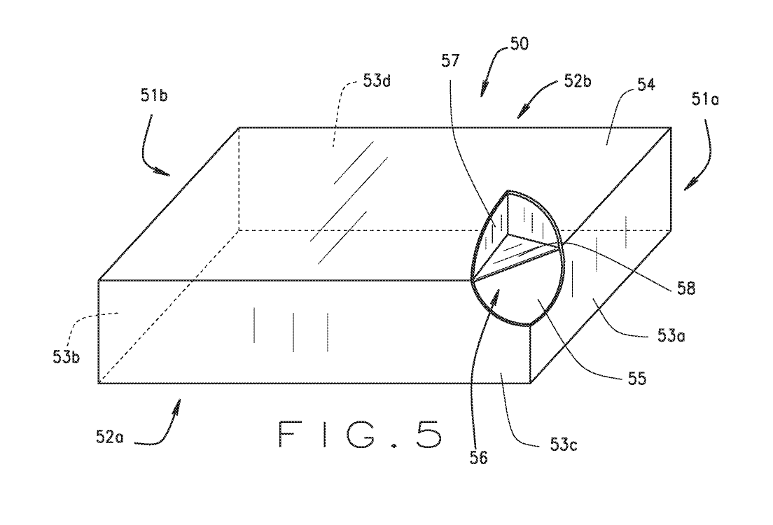

FIG. 4 illustrates yet another alternative dispensing device 40 that is substantially similar to dispensing devices 20, 30 discussed herein above. Dispensing device 40 includes right and left end portions 41a and 41b, respectively. It also includes front and rear portions 42a and 42b, respectively. Dispensing device 40 includes side wall members 43, including each of a right side wall 43a, a left side wall 43b, a front side wall 43c, and a rear side wall 43d. A top member 44 of dispensing device 40 may further include an opening 45 occupies right side portion 41a of top member 44 and the right side portion 41a side wall 43 dispensing device 30. That way, in a manner substantially similar to dispensing device 30, when gloves 46 of dispensing device 40 being dispensed are getting low in supply, it is easy for a user to tell that gloves 46 are in need of replacement. Like dispensing device 20, 30, dispensing device 40 allows for gloves 46 to be removed from dispensing device 40 by cuffs 47 thereof in order to reduce the risk of contamination and/or desterilization.

Dispensing device 40 may be made of materials substantially similar to dispensing devices 20, 30. And also like dispensing devices 20, 30, dispensing device 40 may be used in a disposable or non-disposable manner. Dispensing device 40 may include disposable packaging 48 substantially similar to disposable packaging 28, 38 described above for containing and storing gloves such as gloves 46.

FIG. 5 illustrates yet another alternative embodiment dispensing device 50, substantially similar to dispensing devices 20, 30, 40 described above. Dispensing device 50 includes right and left side portions 51a and 51b, respectively, and front and rear portions 52a and 52b, respectively. Dispensing device 50 also includes four side walls 53a, 53b, 53c, and 53d, and a top member 54. A circular-shaped opening 55 is provided at an intersection 56 of two side walls 53 at a right side portion 51a and a front side portion 52a. Other shapes for opening 55 are contemplated herein. For example, door member may be the entire top member 54. Alternatively, door member 57 may be replaced by an outer shell (not illustrated) for surrounding the devices 20, 30, 40 that is able to open and/or close to reveal opening 25, 35, 45. Circular opening 55 may improve the ability of a user to reach gloves (not illustrated) within dispensing device 50.

As shown in FIG. 5, dispensing device 50 further includes a door member 57. Door member 57 is preferably hingedly attached to top member 54 of dispensing device 50 via hinges 58. Door member 57 may be automatically programmed such that it may be opened to access gloves therein on demand by a user, for example with the press of a button or flip of a switch. Alternatively, door member 57 may be programmed such that when a user waves his or her hand near opening 55, door member 57 automatically opens to access gloves therein. Automated systems for allowing door member 57 to be automatically controlled are well known and understood in the art.

In the alternative, door member 57 may be manually controlled in a manner long known and understood in the art. It should be noted that alternative embodiments of dispensing devices 20, 30, 40 may include a door member substantially similar to door member 57. In those embodiments, a door member would be sized and shaped to cover any of openings 25, 35, 45 when gloves 26, 36, 46 are not being dispensed. In its other respects, dispensing device 50 acts substantially similarly to dispensing devices 20, 30, 40.

From the foregoing, it will be seen that this invention is one well adapted to attain all the ends and objects hereinabove set forth together with other advantages which are obvious and which are inherent to the structure. It will be understood that certain features and sub combinations are of utility and may be employed without reference to other features and sub combinations. This is contemplated by and is within the scope of the claims. Since many possible embodiments of the invention may be made without departing from the scope thereof, it is also to be understood that all matters herein set forth or shown in the accompanying drawings are to be interpreted as illustrative and not limiting.

The constructions described above and illustrated in the drawings are presented by way of example only and are not intended to limit the concepts and principles of the present invention. Thus, there has been shown and described several embodiments of a novel packaging for dispensing of disposable gloves. As is evident from the foregoing description, certain aspects of the present invention are not limited by the particular details of the examples illustrated herein, and it is therefore contemplated that other modifications and applications, or equivalents thereof, will occur to those skilled in the art. The terms “having” and “including” and similar terms as used in the foregoing specification are used in the sense of “optional” or “may include” and not as “required”. Many changes, modifications, variations and other uses and applications of the present construction will, however, become apparent to those skilled in the art after considering the specification and the accompanying drawings. All such changes, modifications, variations and other uses and applications which do not depart from the spirit and scope of the invention are deemed to be covered by the invention which is limited only by the claims which follow.

What is claimed is:

1. A dispensing system for dispensing disposable gloves, the packaging system comprising:
a dispensing device including:
a right side wall, a left side wall, a front side wall, and a rear side wall at each of a right portion, a left portion, a front portion, and a rear portion of the dispensing device; and
a top member including an opening in at least one of the right portion and the left portion of the top member of the dispensing device; and
a plurality of disposable gloves contained within the dispensing device, the plurality of disposable gloves stacked on top of one another, each of the disposable gloves facing substantially the same direction with a cuff end of said plurality of disposable gloves positioned at the opening.

2. The dispensing system of claim 1, wherein the dipensing system further includes a disposable packaging within the dispensing device for containing the plurality of disposable gloves therein.

3. The dispensing system of claim 1, wherein the dispensing system further includes a door member hingedly attached to the top portion of the dispensing device for covering the opening when the dispensing device is not in use.

4. The dispensing system of claim 1, wherein the opening also occupies at least part of one of the front side wall and the rear side wall.

5. The dispensing system of claim 1, wherein the opening also occupies at least part of one of the right side wall and the left side wall.

6. The dispensing system of claim 1, wherein the opening also occupies at least part of the right side wall or the left side wall and at least part of the front side wall and the rear side wall.

7. The dispensing system of claim 1, wherein the packaging inside is disposable.

8. The dispensing system of claim 1, wherein the dispensing system is non-disposable.

9. The dispensing system of claims 2 and 7, wherein the disposable packaging is preferred to be waterproof such as a plastic bag, but non waterproof bag like paper or nonwoven is acceptable when dispensing system is waterproof.

10. The dispensing system of claim 1, wherein the dispensing device is made up of at least one of but not limiting to plastic, metal, wood, rubber, or leather, etc.

11. The dispensing system of claim 3, wherein the door member can be opened manually or automatically when glove dispensing is needed.
